(19) United States
(12) Patent Application Publication
(10) Pub. No.: US 2013/0334418 A1
(43) Pub. Date: Dec. 19, 2013

(54) SMART TUNABLE DIODE LASE MODULE

(71) Applicants: Alan Cowie, Friendswood, TX (US); Jie Zhu, Pearland, TX (US)

(72) Inventors: Alan Cowie, Friendswood, TX (US); Jie Zhu, Pearland, TX (US)

(21) Appl. No.: 13/986,909

(22) Filed: Jun. 17, 2013

Related U.S. Application Data
(60) Provisional application No. 61/690,096, filed on Jun. 19, 2012.

(51) Int. Cl. G01N 33/00

(52) U.S. Cl. CPC G01N 33/0009
USPC 250/339.03; 372/38.1; 250/339.06

(57) ABSTRACT

An improved laser module for a tunable diode laser spectroscopy analyzer. The improvement is a programmable non-volatile memory device (such as an EEPROM device) attached to the module. In addition, an improved method for updating the laser parameters for a tunable diode laser analyzer when a new laser module is installed in the analyzer. The improvement is the step of reading the parameters from a programmable non-volatile memory device (such as an EEPROM device) attached to the module.

This application claims priority to U.S. Provisional Patent Application 61690096 filed 19 Jun. 2012.

The instant invention is in the field of gas analysis, such as combustion gas analysis, and more specifically the instant invention is in the field of tunable diode laser spectroscopic analysis of a gas. A tunable diode laser emits near monochromatic light of a wavelength that is dependent on the current fed to the diode. Tunable diode laser spectroscopic analysis of combustion gases is known and described in the prior art, for example, by: Lackner et al., Thermal Science, V.6, p13-27, 2002; Allen, Measurement Science and Technology, V.9, p545-562, 1998; Nikkary et al., Applied Optics, V.41(3), p446-452, 2002; Upschulte et al., Applied Optics, V.38(9), p1506-1512, 1999; Mihalcea et al., Measurement Science and Technology, V.9, p327-338, 1998; Webber et al., Proceedings of the Combustion Institute, V.28, p407-413, 2000; Ebert et al., Proceedings of the Combustion Institute, V.30, p1611-1618, 2005; Nagali et al., Applied Optics, V.35(21), p4027-4032, 1996; and U.S. Pat. Nos. 7,248,755 7,244936 and 7,217,121.

### BACKGROUND OF THE INVENTION

U.S. Patent Application Publication 2010-0028819 and www.yokogawa.comius/ia/analytical/tdls200 describe the TruePeak® TDLS200 tunable diode laser spectroscopy analyzer from Yokogawa Corporation of America. FIG. 4 of the '819 publication details the laser and detector system. Referring to said FIG. 4 of the '819 publication, the tunable diode laser gas analysis system includes a laser module 37 containing the tunable diode laser. A control unit 31 contains the central processing unit (CPU) programmed for signal processing as well as the temperature and current control for the tunable diode laser and a user interface and display. Alignment plate 29 and adjustment rods 30 allow alignment of the laser beam 41. Dual process isolation windows 28 are mounted in a pipe flange 40. The space between the windows 28 is preferably purged with nitrogen. In this specific example, the flange 40 is mounted through the wall of an industrial furnace. The laser beam 41 is passed through the combustion gas and then through dual process isolation windows 33 to a near infrared light detector 38. The windows 33 are mounted in a pipe flange 39. The space between the windows 33 is preferably purged with nitrogen. The flange 39 is mounted through the wall of the furnace. Alignment plate 34 and adjustment rods 35 allow alignment of the detector optics with the laser beam 41. Detector electronics 36 are in electrical communication with the control unit 31 by way of cable 37a. The control unit 31 is also in electrical communication (by way of electrical cables 38a) with a process control system 32 for controlling the furnace.

The above described system of the '819 publication operates by measuring the amount of laser light at specific wavelengths, which light is absorbed (lost) as it travels through the combustion gas. Carbon monoxide, gaseous water and hydrocarbons each have a spectral absorption of infrared light that exhibits unique fine structure. The individual features of the spectra are seen at the high resolution of the tunable diode laser.

The system described above is commercially successful and is used, for example, to optimize the operation of furnaces in oil refineries. However, the characteristics of the laser diode can drift over time that requires the analyzer to be re-calibrated periodically, say once per two years. Additionally, the laser diode has a specified service life and eventually will need to be replaced either on a periodic preventative maintenance schedule or upon the eventual failure of the laser diode or upon other unknown random failure mode. There are a sufficient number of parameters unique to each laser diode (such as the precise wavelength of the diode at a specific precise current fed to the diode, the slope of wavelength from the diode v. current fed to the diode and the wavelength variation with temperature of the diode) that the analyzer as a whole must be recalibrated when the diode is replaced. If the replacement laser module is not recalibrated the results from the analyzer will be incorrect at best or more probably the analyzer will not even work.

The first step in recalibrating an existing or replacement laser module in the prior art is to remove the complete analyzer including the laser, detector (and controller, depending on analyzer architecture) from the live process and attach it to an off-line calibration cell taking care not to contaminate the process or create a hazardous condition by releasing process gas. The movement of the analyzer from the process to the off-line condition is often restrictive due to the numerous cables, conduits, tubes, pipes and other outdoor site logistics that exist in industrial plant environments. After some initial alignment of the laser and detector, the calibration cell is then flushed with a transparent gas such as nitrogen. Then the calibration cell is flushed with a known gas taking care that there are no gas leaks in the calibration cell. The user must then very carefully enter a series of new parameters at the CPU that are specific to the new laser diode—these are typically the target operating temperature, the tuning range, and any diode specific compensation parameters. These parameters may have been provided in the form of text, or uploadable file or other format but great care must be taken to ensure the correct parameters are entered or uploaded to the analyzer. The pressure, temperature, and path length of the calibration gas is then inputted into the CPU of the analyzer and a system calibration program is initiated to calibrate the system for the new laser module. The laser unit and detector unit (and controller depending on architecture) is then reinstalled on the process. Again, great care must be taken to ensure there is no hazardous gas release and other industrial hazardous are avoided. The user must now input the correct process operating conditions for the process analyzer otherwise the measurement values will be incorrect. Such recalibration is especially difficult because it must be done on site in an industrial environment. Furthermore, an unforeseen failure generally requires immediate action from the user to ensure the analyzer is returned to service as soon as possible—the current method does not lend itself well to these situations. It would be an advance in the art if a means were discovered that would allow a diode laser module to be replaced without the need to then recalibrate the analyzer on site and/or having to perform additional parameter setting changes or system updates.

### SUMMARY OF THE INVENTION

The instant invention is a solution to the above-mentioned problem. The instant invention is an improved smart diode laser module, the module containing the diode and a programmable non-volatile memory device (such as an EEPROM chip) attached to the module. Diode specific information (such as the above mentioned wavelength v. current) is pre-stored in the non-volatile memory. The smart module of the instant invention facilitates plug-in style assembly when the analyzer is manufactured and when the analyzer is serviced in use. The instant invention provides for the replacement of the laser module of a tunable diode laser spectroscopy analyzer without the need to recalibrate the analyzer on site. If desired, a spare laser module can be installed while the original laser module is validated off-line.

### BRIEF DESCRIPTION OF THE DRAWINGS

FIG. 1 is a side view, part in cross-section and part in full, of an embodiment of the instant invention showing a laser module comprising a programmable non-volatile memory device;

FIG. 2 is a block diagram of a tunable diode laser spectroscopy analyzer system wherein the laser module comprises a programmable non-volatile memory device;

### DETAILED DESCRIPTION

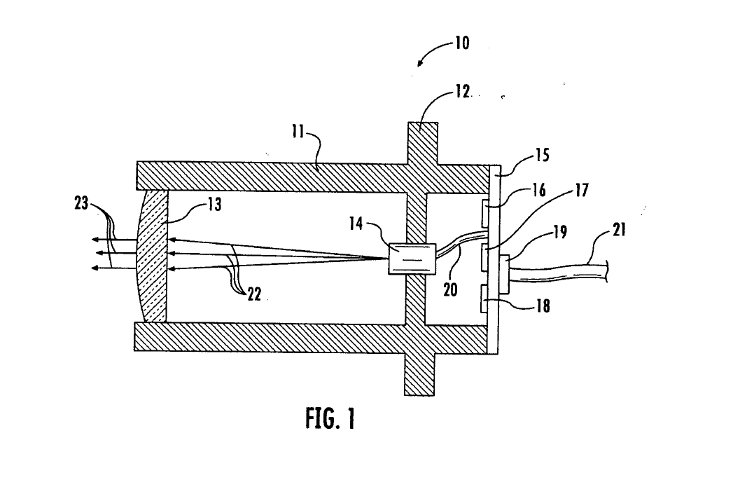

Referring now to FIG. 1, therein is shown a preferred improved laser module 10 of the instant invention part in cross section and part in full. The laser module 10 comprises a tubular body 11 having a mounting flange 12. A collimation lens 13 is mounted at one end of the body 11. A circuit board 15 is mounted at the other end of the body 11. A tunable diode laser 14 is mounted in the body 11 and is in electrical communication with circuit board 15 by way of diode cable 20. The laser 14 emits a beam of light 22, which beam is collimated as a collimated beam of light 23 by collimation lens 13. An electrical connector 19 is mounted on the circuit board 15. A CPU cable 21 is connected to the connector 19. An electronic filter 16 is mounted on the circuit board 15 in electrical communication with the laser 14. A temperature sensor 17 is mounted on the circuit board 15 in electrical communication with the CPU cable 21. An EEPROM chip 17 is mounted on the circuit board 15 in electrical communication with the CPU cable 21.

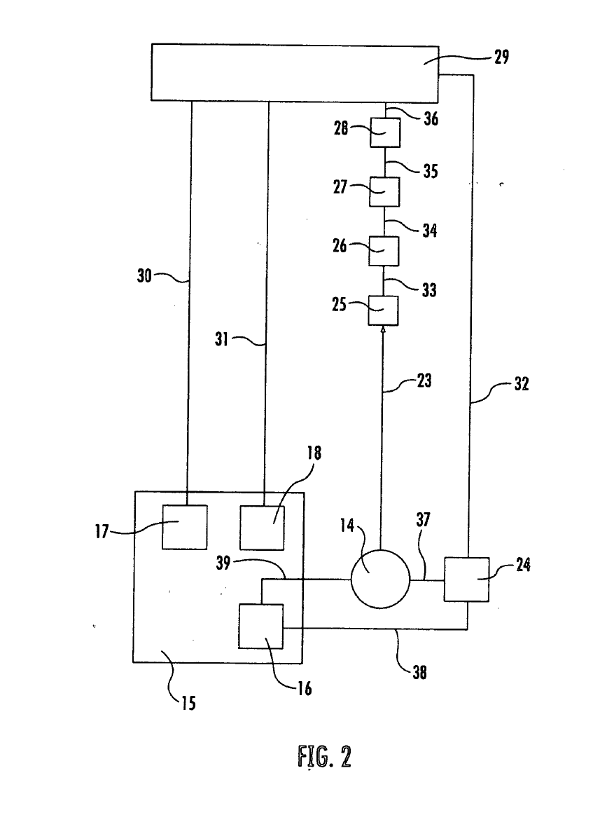

Referring now to FIG. 2, therein is shown a block diagram of a tunable diode laser spectroscopy analyzer system including tunable diode laser 14. Light 23 from the laser 14 is shown through the gas to be analyzed to a light detector 25. The signal from the light detector 25 is directed to a current to voltage converter 26 by detector cable 33. The signal from the current to voltage converter 26 is directed to a voltage amplifier 27 by amplifier cable 34. The signal from the offset amplifier 27 is directed to an analog to digital converter 28 by a/d input cable 35. The digital signal from the analog to digital converter 28 is directed to a central processing unit (CPU) 29 by way of aid output cable 36. The laser 14 is driven by electrical current from electronic filter 16 by way of filter output cable 39. The filter 16 is mounted on circuit board 15. The electronic filter 16 receives current from laser control circuit 24 by filter input cable 38. The laser control circuit 24 is in electronic communication with CPU 29 by way of laser control cable 32. A temperature sensor 17 is in electronic communication with the CPU 29 by temperature sensor cable 30. The signal from the temperature sensor 17 is processed by CPU 29 and directed to laser control circuit 24 to control the temperature of laser 14 by way of a peltier device attached to the laser 14. The peltier device is powered by the laser control circuit 24 by way of peltier cable 37. The above so far described system of FIG. 2 is commercially available prior art available from Yokogawa Corporation of America as the TruePeak® TDLS200 tunable diode laser analyzer. What follows is the improvement of the instant invention to the laser module of this or any other TDL analyzer that incorporates a laser module. The improvement is to attach a programmable non-volatile memory device to the laser module. Preferably the programmable non-volatile memory device is the EEPROM chip 18. The EEPROM chip 18 is in electronic communication with the CPU 29 by EEPROM cable 31. Data stored in the EEPROM chip 18 preferably includes the laser module serial number, laser temperature control parameters, laser current drive parameters, laser power information (including a power spectrum with zero gas absorption) and span calibration coefficients and absorption spectrum. Each time the tunable diode laser spectroscopy analyzer is powered up, the CPU 29 reads the data from the EEPROM chip 18. If a new laser module is identified the information from the EEPROM chip 18 is used to automatically update the operation of the analyzer 42.

### EXAMPLE

This example demonstrates the replacement of a laser module of a tunable diode laser gas analyzer installed on an industrial furnace with significantly reduced difficulties of working in an industrial environment. In the comfort of a electronic laboratory, a new TruePeak TDLS200 tunable diode laser module from Yokogawa Corporation of America is modified by attaching an EEPROM chip to the circuit board of the module so that the EEPROM chip is in electronic communication with the CPU cable connector on the back of the module. The laser module is attached to one side of a calibration cell. A TruePeak TDLS200 tunable diode laser analyzer detector is attached to the other side of the calibration cell. The calibration cell is then flushed with nitrogen and the detector is connected to a TruePeak TDLS200 tunable diode laser analyzer. The alignment of the laser module is then optimized so that the raw detector signal is flat for the first 20 data points. Then the calibration cell is flushed with a mixture containing a known concentration of carbon monoxide, carbon dioxide, water, methane and oxygen, taking care that there are no gas leaks in the calibration cell. The pressure, temperature, and path length of the calibration gas is inputted into the CPU of the analyzer and a system calibration program is initiated to calibrate the system for the first laser module. The EEPROM chip of the laser module is programmed by the CPU of the analyzer for the laser module serial number, the laser temperature control parameters, the laser current drive parameters, the laser power information (including a power spectrum with zero gas absorption) and the span calibration coefficients and absorption spectrum for carbon monoxide, methane and water. The new module is removed from the calibration cell and shipped to a location having a TruePeak TDLS200 tunable diode laser analyzer mounted on an industrial furnace, the original laser module of which analyzer needs replacement. The analyzer is powered off, the original laser module is removed from the analyzer and the new laser module is installed on the analyzer. The analyzer is powered on. The CPU of the analyzer reads the laser module serial number, the laser temperature control parameters, the laser current drive parameters, the laser power information (including a power spectrum with zero gas absorption) and the span calibration coefficients and absorption spectrum for carbon monoxide, methane and oxygen from the EEPROM chip of the new module.

### CONCLUSION

While the instant invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claim.

What is claimed is:

1. An improved laser module for a tunable diode laser spectroscopy analyzer, wherein the improvement comprises a programmable non-volatile memory device attached to the module.

2. The improved module of claim 1 wherein the programmable non-volatile memory device is an EEPROM device.

3. The improved module of claim 1, wherein the programmable non-volatile memory device is programmed with the laser module serial number, the laser temperature control parameters, the laser current drive parameters, the laser power spectrum, zero gas absorption, span calibration coefficients and absorption spectrum for carbon monoxide, methane and oxygen.

4. The improved module of claim 2, wherein the programmable non-volatile memory device is programmed with the laser module serial number, the laser temperature control parameters, the laser current drive parameters, the laser power spectrum, zero gas absorption, span calibration coefficients and absorption spectrum for carbon monoxide, methane and oxygen.

3. An improved method for updating the laser module serial number, the laser temperature control parameters, the laser current drive parameters, the laser power spectrum, zero gas absorption, span calibration coefficients and absorption spectrum for carbon monoxide, methane and oxygen for a tunable diode laser analyzer when a new laser module is installed in the analyzer, wherein the improvement comprises the step of reading the laser module serial number, the laser temperature control parameters, the laser current drive parameters, the laser power spectrum, zero gas absorption, span calibration coefficients and absorption spectrum for carbon monoxide, methane and oxygen from a programmable non-volatile memory device attached to the module.

4. The improved method of claim 3, wherein the programmable non-volatile memory device is an EEPROM device.

5. An improved tunable diode laser gas analyzer comprising a laser module, wherein the improvement comprises a programmable non-volatile memory device attached to the module.

6. The improved analyzer of claim 5 wherein the programmable non-volatile memory device is an EEPROM device.

7. The improved analyzer of claim 5, wherein the programmable non-volatile memory device is programmed with the laser module serial number, the laser temperature control parameters, the laser current drive parameters, the laser power spectrum, zero gas absorption, span calibration coefficients and absorption spectrum for carbon monoxide, methane and oxygen.

8. The improved analyzer of claim 6, wherein the programmable non-volatile memory device is programmed with the laser module serial number, the laser temperature control parameters, the laser current drive parameters, the laser power spectrum, zero gas absorption, span calibration coefficients and absorption spectrum for carbon monoxide, methane and oxygen.
